(19) United States
(12) Patent Application Publication
(10) Pub. No.: US 2007/0191686 A1
(43) Pub. Date: Aug. 16, 2007

(54) Method of making a surgical clamp

(75) Inventors: Todd W. Sharratt, Stillwater, MN (US); Christopher Lee Berg, Anoka, MN (US); Steven Karl Jacobson, St. Paul, MN (US); Kevin Rodby, Shoreview, MN (US)

Correspondence Address: SHEWCHUK IP SERVICES, 533 77TH STREET WEST, EAGAN, MN, 55121, US

(73) Assignee: LeVahn Intellectual Property Holding Company, LLC, St. Paul, MN (US)

(21) Appl. No.: 11/352,883

(22) Filed: Feb. 13, 2006

(51) Int. Cl. A61B 1/32

(52) U.S. Cl. 600/227

(57) ABSTRACT

A surgical clamp includes a support frame clamp member, a retraction shaft clamp member and a handle linked to the support frame clamp member and the retraction shaft clamp member for moving the clamp members between loosened and tightened positions. The handle includes a cam, and the handle/cam is unitarily formed by metal injection molding. The handle includes relatively uniform thickness throughout to better accept the shrinkage induced by the metal injection molding process. The base used for a bottom position of the cam is adjustable and spring loaded, to better support a wider range of manufacturing tolerances on the cam as well as the other components of the clamp without over stressing the metal injection molded handle during use. The handle includes a gripping portion with a planar portion inducing alignment of the clamping force as desired for best operation of the clamp.

### CROSS-REFERENCE TO RELATED APPLICATION(S)

None.

### BACKGROUND OF THE INVENTION

The present invention relates to the field of surgical tools, and particularly to the design and manufacture of surgical retractor systems. Surgical retractor systems are used during surgery to bias and hold tissue in a desired position. As one example, some surgical procedures require anterior access to the spine, through the patient's abdomen. Tissue such as skin, muscle, fatty tissue and interior organs needs to be held retracted to the side so the surgeon can obtain better access to the vertebrae structures of primary interest.

Surgical retraction may be performed by one or more aides using handheld tools, with the most basic retractor apparatus being a tongue depressor. More commonly now in sophisticated operating rooms during abdominal or chest surgery, a surgical retractor system or assembly is used. The retractor assembly may, for instance, include a ring or support frame which is rigidly supported from the patient's bed above and around the surgical incision location, with a number of clamps and retractor blades to hold back tissue proximate to the surgical incision. Other retraction systems, such as those disclosed in U.S. Pat. Nos. 6,315,718, 6,368,271 and 6,659,944 to Sharratt, incorporated herein by reference, may not include a ring and/or may be directed at other types of surgery. Clamps may also be used to attach the ring or support frame to a support post and/or part of the bed frame.

One style of surgical clamp which has gained some marketplace acceptance includes a handle which moves a cam or wedge to effect the clamping force for the clamp. Examples include the surgical clamps disclosed in U.S. Pat. Nos. 5,727,899, 5,792,046, 5,888,197, 5,899,627, 6,017,008, 6,042,541 and 6,264,396. The handle provides a torque through a pivoting action, which generally provides a great mechanical advantage to the clamp. For instance, a handle throw of several inches may result in a cam movement of several hundredths of an inch, i.e., a mechanical advantage on the order of 10² or more. Though the handle throw force may be only 10 to 50 pounds, the forces and torques sustained by the handle and cam may be considerable, providing the most likely location for clamp failure. In that surgical clamps are used in critical surgery applications, inopportune clamp failure is not a permissible risk. Such handles, and their associated cams or wedges, are typically machined out of stainless steel bar stock and subsequently heat treated, such as a 17-4 stainless steel, precipitation hardened and heat treated to condition H 900.

In devising a proper clamping structure, the clamp should give the surgeon flexibility in quickly assembling the retraction system and in placement of the various retractors. (The term “surgeon” is used herein including the person operating the clamp, who may or may not be the person performing the actual surgery.) Once the various retractors are in place and oriented and pulled as desired, the retraction system clamps should allow quick and easy tightening so the entire retraction system is maintained fixedly in place. Once tightened the retraction system should be unobtrusive so neither the tissue held retracted nor the retraction system interfere in any way with the surgeon or the surgical procedure. After surgery is completed (or perhaps once or more during surgery), the retraction system should quickly loosen and/or disassemble so as relax the retracted tissue and minimize damage to the retracted tissue. Surgical retractor systems must be robust and strong, as even a slight possibility of failure during use is not tolerated. Surgical retractor assemblies should be readily reusable, including sterilizable, for use in multiple surgeries. Surgical retractor systems should maintain a relatively low cost. Improvements in surgical retractor clamps and systems can be made in keeping with these goals.

### BRIEF SUMMARY OF THE INVENTION

The present invention is a surgical clamp and clamping system using a handle, which, in one aspect, is unitarily formed with the wedge or cam member. The handle/cam is not machined from bar stock or from a casting, but rather is metal injection molded. The metal injection molding process includes a significant shrinkage during manufacture. In one aspect, the handle utilizes the benefits of the metal injection molding process in providing a shaped handle with a double offset, while avoiding non-uniform shrinkage by providing a design with largely uniform thicknesses and volumes. In another aspect, dimensional tolerances on the cam member are increased by having a separate, post-initial assembly manufacturing adjustment for the distance through which the camming action is applied on the other parts of the clamp design.

### BRIEF DESCRIPTION OF THE DRAWINGS

FIG. 1 is a perspective view of a preferred surgical clamp in accordance with the present invention.

FIG. 2 is an exploded perspective view of the clamp of FIG. 3.

FIG. 3 is an elevational side view of the clamp of FIGS. 1 and 2, showing the loosened position of the handle in dotted lines.

FIG. 4 is a plan view of the clamp of FIGS. 1-3, showing the range of adjustment of the shaft and handle relative to the support frame.

FIGS. 5 and 6 are end views of the clamp of FIGS. 1-4.

FIG. 7 is an opposing elevational side view of the clamp of FIGS. 1-6 in the loosened position.

FIG. 8 is a side view of the clamp of FIG. 7 in the tightened position.

FIG. 9 is a finite element analysis rendering of the handle/cam of FIGS. 1-8.

FIG. 10 is a finite element analysis rendering of the cam of FIGS. 1-8.

While the above-identified drawing figures set forth one or more preferred embodiments, other embodiments of the present invention are also contemplated, some of which are noted in the discussion. In all cases, this disclosure presents the illustrated embodiments of the present invention by way of representation and not limitation. Numerous other minor modifications and embodiments can be devised by those skilled in the art which fall within the scope and spirit of the principles of this invention.

### DETAILED DESCRIPTION

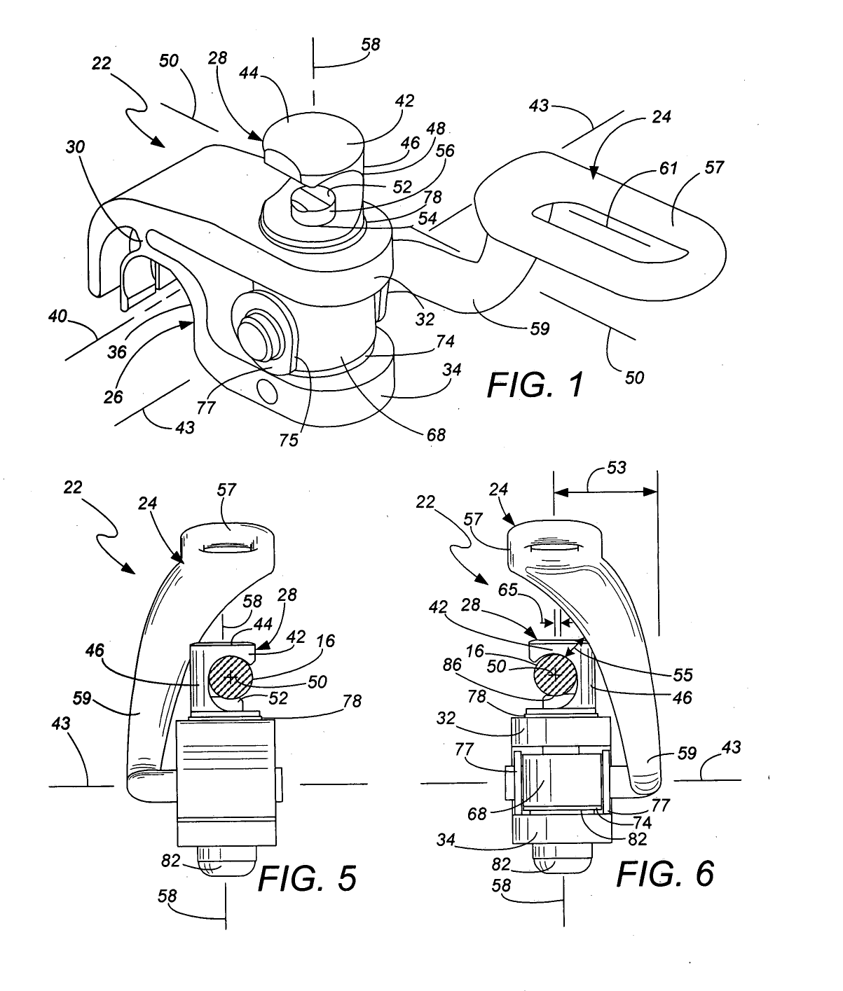

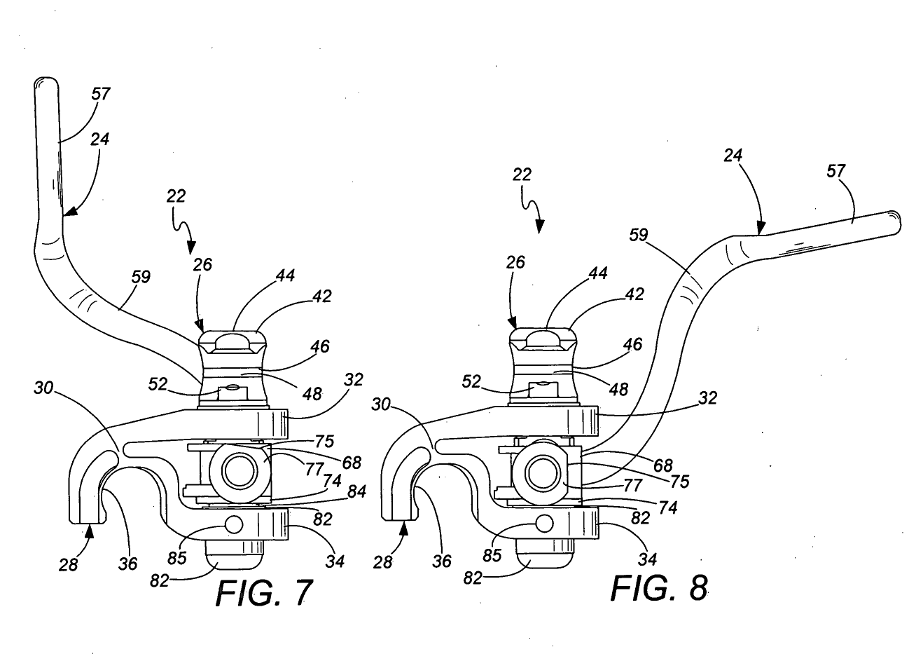

The method and apparatus of the present invention will be described with reference to a clamp member 22 as further disclosed in Application No. M1.12-4, incorporated by reference. The clamp 22 primarily includes a tightening handle 24, a first clamp member 26 (in the lower position as shown in FIGS. 1-8, it being recognized that orientation of the clamp 22 may depend upon use) and a second clamp member 28 (in the upper position as shown in FIGS. 1-8). For ease of description, the first or lower clamp member 26 will be called a “frame” clamp and the second or upper clamp member 28 will be called a “shaft” clamp, recognizing that the first clamp 26 may attach to a rod other than the support frame 18 and the second clamp 28 may attach to a rod other than a retractor shaft 16.

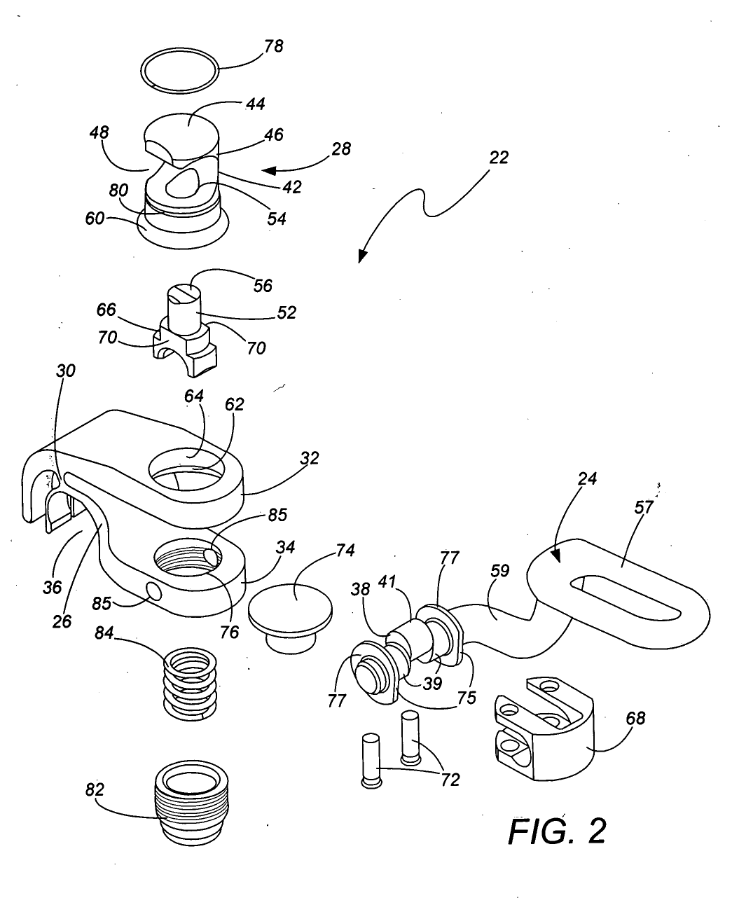

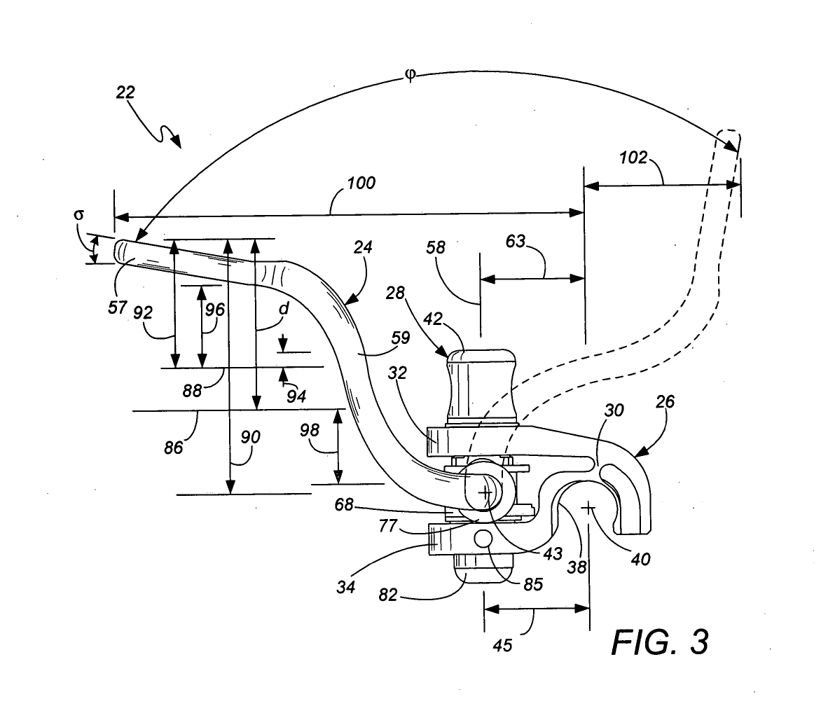

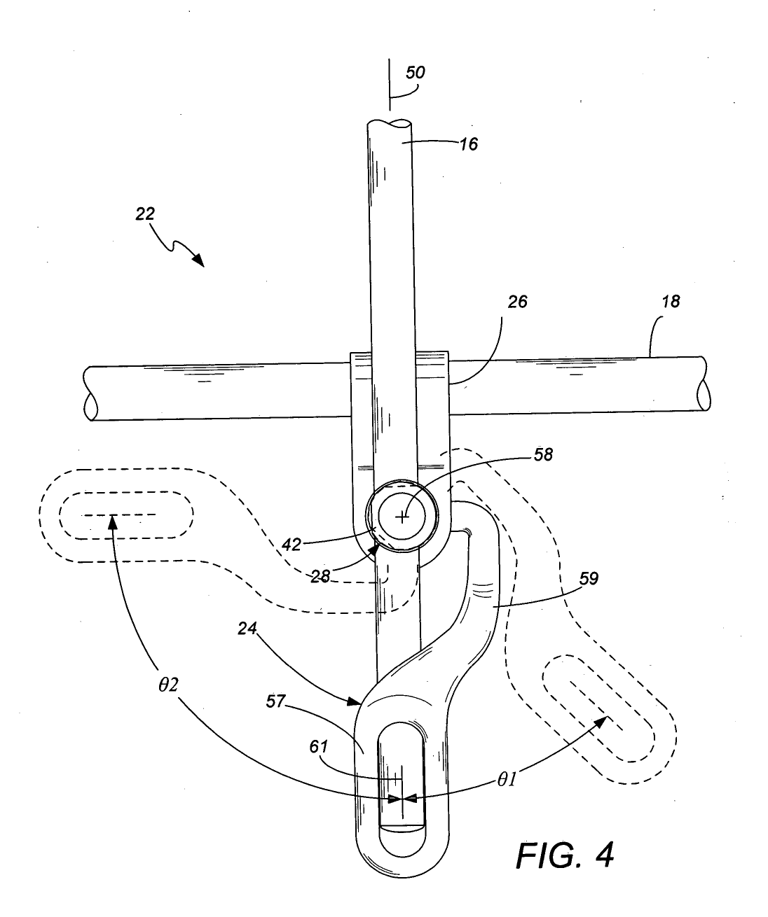

The frame clamp 26 may be a fulcrum clamp as generally disclosed in U.S. Pat. No. 5,727,899 and in application Ser. Nos. 10/664,195 filed Sep. 17, 2003 and 11/330,625 filed Jan. 12, 2006, all incorporated by reference. The preferred fulcrum clamp 26 thus includes a fulcrum portion 30 extending between an upper leg portion 32 and a lower leg portion 34. The fulcrum portion 30 allows the size of the frame clamp opening 36 to change based upon biasing the upper leg portion 32 away from the lower leg portion 34. A wedge or cam 38 (shown in FIG. 2) positioned between the upper and lower leg portions 32, 34 is movable to force the upper and lower leg portions 32, 34 apart and causes the fulcrum portion 30 to flex. As the fulcrum portion 30 flexes, the frame clamp opening 36 constricts. When the frame clamp opening 36 constricts, the clamp 22 can frictionally attach onto the support frame 18. The frame clamp opening 36 extends longitudinally on the frame 18 for a sufficient distance to define a rod axis 40 (shown in FIGS. 1 and 3) and orientation of the frame 18 (FIG. 4) in the frame clamp opening 36. In the preferred embodiment, the frame clamp 26 is about ¾ inch wide. The frame clamp opening 36 is sized to mate with the cross-sectional size and shape of the support frame 18, such as a ½″ diameter cylindrical shape. In the loosened position, the frame clamp 26 permits substantially unimpeded longitudinal movement of the clamp 22 on any linear portion of the support frame 18, as well as substantially free rotation of the clamp 22 about the support frame axis 40.

The clamping force is provided by a wedge or cam member 38 placing equal and opposite forces on the upper and lower leg portions 32, 34. Once the frame clamp 26 is closed to a tightened position, it does not require further application of force or holding by the surgical staff to remain in the tightened position. The preferred frame clamp cam 38 includes two outer lobes 39 for pushing downward and a central lobe 41 for pushing upward so it can provide a balanced force and for ease of manufacture and assembly. In the preferred embodiment, both the central lobe 41 and the outer lobes 39 are circular with diameters of about ¼ of an inch, and with the axis of the central lobe 41 offset from the axis of the outer lobes 39 by about ⅛ of an inch.

As best shown in FIGS. 1, 2, 5 and 6, the shaft clamp 28 primarily includes a stanchion head 42 having a top stanchion 44 extending rigidly from a side stanchion 46. The inside surfaces of the top stanchion 44 and side stanchion 46 are sized and shaped to mate with the retractor shaft 16. The shaft clamp opening 48 extends longitudinally on the shaft 16 for a sufficient distance to define a rod axis 50 and orientation of the shaft 16 in the shaft clamp opening 48. The shaft clamp opening 48 also defines the placement of the bottom and top surfaces 86, 88 of the shaft 16. In the preferred embodiment, the shaft clamp 28 is about inch wide. The shaft clamp opening 48 is sized to mate with the cross-sectional size and shape of the shaft 16, such as a ⅜″ diameter cylindrical shape. In the loosened position, the shaft clamp 28 permits substantially unimpeded longitudinal movement of the shaft 16 in the shaft clamp opening 48, as well as substantially free rotation of the shaft 16 about its axis 50 in the shaft clamp opening 48.

The shaft clamp 28 is preferably activated by the same handle 24 as the frame clamp 26. To achieve the simultaneous tightening with a single handle 24, pivoting movement of the handle 24 not only causes the wedge 38 to increase separation between the upper and lower legs 32, 34, but also moves a pin 52 vertically upward to press the retractor shaft 16 against the top stanchion 44. The pin 52 translates or slides in a pin bore 54 in the bottom of the shaft clamp 28. The pin bore 54 intersects the shaft clamp opening 48, so the pin 52 can be biased against the outer profile of the shaft 16 by sliding the pin 52 in the pin bore 54.

The handle 24 is oriented to the side with a horizontal offset 53 providing a minimum clearance 55 (best shown in FIG. 6) relative to the retractor shaft 16 to further facilitate access during surgery to quickly and easily snap the shaft 16 into the clamping opening 48. The handle 24 includes a grasping portion 57 and an arm portion 59 extending from the cam 38. The grasping portion 59 is centered relative to the shaft axis 50, so biasing the handle 24 tighter or looser by the surgeon will result in no net rotational moment of the clamp 22 about the shaft axis 50. In the preferred embodiment as shown in FIGS. 1 and 4, the grasping portion 57 defines a plane with a grasping centerline 61 vertically in line with the shaft axis 50. The planar nature of the grasping portion 57 helps induce the surgeon to apply the tightening force centered within and normal to the grip plane, which is thereby also normal to the handle pivot axis 43 and centered relative to the cam 38, such that a naturally oriented tightening force on the handle 24 provides substantially 100% tightening moment, while minimizing non-beneficial stress on the handle 24 and non-beneficial stress on the connection between the frame clamp 26 and the frame 18.

The shaft clamp 28 is preferably pivotable relative to the frame clamp 26 about the vertical axis 58. To achieve the pivoting feature, the shaft clamp 28 is attached to the frame clamp 26 through a rotatable attachment. After the clamp 22 is positioned on the support frame 18 and the shaft 16 is positioned in the shaft clamp 28 but before the handle 24 is moved from the loosened position to the tightened position, the shaft 16 is pivotable about the pivot axis 58. As shown in FIG. 3, an offset 63 between the frame axis 40 and the pivot axis 58 for the shaft 16 is as small as possible, e.g., axis 40 and axis 58 would intersect in the ideal design. In the preferred embodiment, the vertical pivot axis 58 for the shaft 40 intersects the horizontal pivot axis 43 for the handle 24 and cam 38, and thus has an offset 63 from the frame opening axis 40 of only about ⅞^{th} of an inch.

As best shown in FIG. 4, the pivot axis 58 for the shaft 16 is preferably centered to bisect the frame clamp 28. As best shown in FIG. 6, an offset 65 between the pivot axis 58 for the shaft 16 and the shaft longitudinal axis 50 is as small as possible. In the preferred embodiment, the offset 65 between the shaft pivot axis 58 and the shaft longitudinal axis 50 is only about 1/20^{th} of an inch. Having an offset 65 between the shaft pivot axis 58 and the shaft longitudinal axis 50 of less than ½ inch or so provides a more balanced feel to the surgeon during pivoting of the retractor shaft 16, because the retractor shaft 16 seems to pivot rather than swing in an arc about the shaft pivot axis 58. While the offset 65 could be eliminated by either making a larger stanchion head 42, by selecting a stronger material for the stanchion head 42 and making the side stanchion 46 smaller, or by positioning the stanchion head 42 (and particularly the side stanchion 46) further from the pivot axis 58, the preferred design is very compact and tight, and the 1/20^{th} of an inch size of the offset 65 is negligible to the perception of most users.

In most surgical procedures and as depicted in the figures (particularly FIG. 4), pivoting of the shaft 16 will take the shaft 16 through a shaft travel path which, in the preferred embodiment, is a substantially horizontal plane. The handle movement direction (i.e., the surface defined by movement of the grasping axis 61 during tightening/loosening pivoting of the handle 24) is preferably at a substantial angle to the shaft travel path, such as perpendicular to the horizontal shaft travel plane.

As best shown in FIG. 2, the preferred rotatable attachment has a frustroconical bottom flange 60 formed integrally with the side stanchion 46. The frustroconical bottom flange 60 mates with a frustroconical recess 62 in a bore 64 within the upper leg portion 32 of the frame clamp 26. The frustroconical nature of this mating relationship permits pivoting of the shaft clamp 28 relative to the frame clamp 26 so long as the handle 24 is in the loosened position, but frictionally prevents pivoting of the shaft clamp 28 relative to the frame clamp 26 once pressure is applied by the handle 24 in the tightened position.

The handle 24 is keyed to the shaft clamp 28 so the handle 24 moves with the shaft clamp 28 and controls the pivoting location of the shaft clamp 28 about the vertical axis 58. The preferred keying structure is through the tightening pin 52, best shown in FIG. 2. A proximal end of the tightening pin 52 includes a non-circular yoke 66, and the central lobe 41 of the cam 38 resides within the yoke 66. A non-circular opening (not shown) in the bottom of the stanchion head 42 mates with and receives the non-circular yoke 66. The preferred non-circular yoke 66 has flats 70 to drive this rotational coupling. When the handle 24 is pivoted about the vertical axis 58, the cam 38 pivots as part of the handle 24, causing both the non-circular yoke 66 and the keyed stanchion head 42 to also pivot. After the clamp 22 is on the support frame 18 and while the handle 24 is still in the loosened position, the surgeon can easily select and adjust the orientation of the shaft clamp 28 in two ways: either by grasping the handle 24 and pivoting the handle 24 about the pivot axis 58, or by placing the retractor shaft 16 in the shaft clamp opening 48 and lightly pivoting the retractor shaft 16 about the pivot axis 58. Either way, the handle 24 always stays oriented in a vertical plane in line with the retractor shaft 16. If desired, the handle plane (defined by the movement of the grasping axis 61 during tightening/loosening pivoting of the handle 24) could alternatively be offset somewhat from the retractor shaft axis 50, preferably remaining at least parallel to the retractor shaft axis 50 if not aligned with the retractor shaft axis 50.

The preferred clamp 22 permits pivoting of the shaft 16 relative to the support frame 18 through angles θ1 and θ2 (best shown in FIG. 4) before the handle 24 interferes with the frame clamp 26 on either side. These large pivoting angles θ1 and θ2 permit great flexibility for the surgeon to determine the best angle for placement of the shaft 16. In the preferred embodiment, the counter-clockwise (from above) pivoting angle θ1 is about 45°, which will accommodate the desired angle of securement for the shaft 16 in the vast majority of applications. This high pivoting angle θ1 is achieved by the horizontal offset 53 together with the shape of the arm portion 59 of the handle 24, which avoids contact between the handle 24 and either the shaft 16 or the frame clamp 26 throughout the complete throw of the handle 24 between the loosened position and the tightened position.

The clockwise pivoting angle θ2 is even greater, and in the preferred embodiment extends about 90° before the proximal end of the handle 24 (the end of the handle 24 beyond the cam 38) interferes with the frame clamp 26. If desired, the length of the proximal end of the handle 24 could be made shorter or the offset 63 increased slightly to permit an even greater clockwise pivoting angle θ2. With the full pivoting angle θ1+θ2 being greater than 180°, any desired angle of securement is possible. For instance, if the surgeon desires to secure the shaft 16 at a 60° counterclockwise angle to the frame 18, the handle 24 could be rotated 120° clockwise and the shaft could be snapped into the shaft clamp 28 in a “backwards” orientation, with the handle 24 tightening toward the surgical arena.

To provide the desired base position for the outer cam lobes 39, the bearing surface between the cam lobes 39 and the lower leg 34 of the frame clamp 26 is provided by a variable height assembly with an adjustable base height, which includes a C-bearing 68, a plunger base 74, a spring 84 and an adjustment plug 82. The plunger base 74 and C-bearing 68 ride on the spring 84, which maintains a loosened compressive force (typically only a few pounds) biasing the assembly upwards. Transverse insertion of a properly-sized retractor shaft 16 into the shaft clamp 28 moves the pin 52 slightly downward, which in turn moves the C-bearing 68 and plunger base 74 slightly downward, against this spring force. During tightening of the clamp 22, first the cam action absorbs the spring deflection until the spring 84 bottoms out. After the spring 84 bottoms out, the remainder of the cam action causes a force loop which: a) forces the pin 52 upward to clamp the shaft 16 against the top stanchion 44, transferring the cam force through the shaft 16 to the stanchion 42, which in turn b) forces the bottom flange 60 upward to clamp the shaft clamp 28 against rotation against the recess 62 of the upper leg portion 32, transferring the cam force to the upper leg portion 32, which in turn c) forces the frame clamp 26 closed by bending at the fulcrum portion 30, to clamp the frame 18 against the lower leg portion 34. The C-bearing 68 follows the outer cam lobes 39, in an arc relative to the yoke 66 and frame clamp 26, during the entire throw of the handle 24.

During assembly of the preferred clamp 22 as best understood with reference to FIG. 2, the stanchion head 42 is inserted into the bore 64 of the upper leg portion 32 from below. An opening 76 in the lower leg portion 34 may be used for access to assist in machining of the upper leg portion 32 and to assist in placement of the stanchion head 42 into the bore 64 from below. An annular groove 80 on the stanchion head 42 is positioned above an upper surface of the upper leg portion 32. Once assembled in position, the stanchion head 42 is then loosely secured to the frame clamp 26 with a snap ring 78. The snap ring 78 is disposed within the annular groove 80 and rotatably holds the stanchion head 42 within the upper leg portion 32 of the frame clamp 26. The pin 52 including the non-circular yoke 66 is then positioned into its keyed opening 54 in the stanchion head 42.

The plunger base 74 is positioned into the lower leg portion 34 from above the lower leg portion 34. The handle 24 is then assembled in place from the side. The handle 24 is first positioned in the C-bearing 68, and retained in position in the C-bearing 68 with two captivation pins 72. Side ears 77 on the cam 38 maintain the cam 38 centered side to side in the C-bearing 68. Once in position, the C-bearing 68 provides stops which limit the throw of the cam 38 in both loosening and tightening to the desired 95° throw angle φ. The keyed yoke 66 is placed over the central lobe 41 of the cam 38 as the handle 24 and C-bearing 68 are slid in from the side, and the upper arm 32 must be sufficiently spaced from the lower arm 34 to enable this side entry of the handle/C-bearing underneath the keyed yoke 66. Side ears 77 may include flats 75 so side ears 77 do not interfere with the upper arm 32 in the loosened position.

Assembly is completed by placing a spring 84 and screwing an adjustment plug 82 in from below to bias the plunger base 74 upward. The adjustment plug 82 is raised a sufficient distance to lift the plunger base 74 enough to prevent the handle/C-bearing from withdrawing out of the keyed yoke 66. Spring 84 preferably has a low spring constant (such as 24 pounds per inch), so it will be fully compressed with a relatively small compression force (such as 4 pounds). The final elevation of the adjustment plug 82 is selected by screwing an appropriate amount to provide the desired loosened and tightened (with spring 84 fully compressed) bottom position spacing for the clamp 22. The terms “elevation” and “bottom position” as used herein refer to a baseline position and direction for the cam action to generate the clamping force (in the preferred embodiment, reached upon bottoming out the spring 84), without regard for the orientation of the clamp 22. For instance, the elevation of the adjustment plug 82 may be set such that a throw force on the handle 24 of 20 pounds will complete the tightening action about an appropriately sized shaft 16 and frame 18. Once the desired elevation for the adjustment plug 82 is achieved, the adjustment plug 82 is set at this elevation by upsetting the threads of the adjustment plug 82 through by using an orbital riveting machine through the holes 85 in the bottom arm 34.

Because the final seated position of the adjustment plug 82 is not set until after all the component parts are fully manufactured and assembled, the tightening force on the handle 24 is not changed by differing dimensions of the component parts within tolerance. That is, all the clamps 22 manufactured can be set to have the same tightening force, even if, for instance, the cam 38 on one clamp 22 is a mil or two larger than the cam 38 of a different clamp 22. The spring 84 places a vertically oriented force on the assembly and, together with proper tightening of the adjustment plug 82, prevents any separation or rattling of parts which might otherwise occur if the dimensional tolerances on any of these parts are not strictly met.

The preferred clamp 22 accordingly permits a loosened attachment to both the support frame 18 and the retractor shaft 16 which has five degrees of freedom: the clamp 22 can be slid longitudinally on the support frame 18; the clamp 22 can be rotated about the longitudinal axis 40 of the support frame attachment portion; the shaft 16 can be pivoted about the vertical axis 58; the shaft 16 can be slid longitudinally in the shaft clamp 28; and the shaft 16 can be rotated about the longitudinal axis 50 of the shaft attachment portion. When the handle 24 is “thrown” or pivoted from the loosened position to its tightened position, all five of these degrees of freedom are secured. During tightening, both the shaft opening and the frame opening dimensions are slightly decreased to eliminate any rotation or translation of the shaft 16 and frame 18 relative to the clamp 22. At the same time, the frictional engagement of the mating frustroconical surfaces 60, 62 after tightening prevent further pivoting of the shaft clamp 28 relative to the frame clamp 26.

The linkage between the handle 24 and the frame clamp 26 and the shaft clamp 28 is fully operated between the fully loosened position and the fully tightened position by a pivoting of the handle 24 through a tightening throw range φ shown in FIG. 3. In contrast to prior art devices, the entire tightening range φ for the clamp 22 is targeted to conform to the size and motion appropriate for a surgeon's hand. To tighten the clamp 22, the surgeon need only grasp the bottom biasing surface 86 (height shown in FIG. 3) of the shaft 16 with the surgeon's fingers and the grip portion 57 of the handle 24 with the surgeon's thumb on the same hand, and squeeze similar to operating a scissors. This scissors squeezing motion is very intuitive, as students have been taught to use scissors since kindergarten. Thus, the top of the grasping portion 57 of the handle 24 is always aligned with and is generally facing away from the bottom biasing surface 86 of the shaft 16. In particular, the grasping surface 57 of the handle 24 should remain within about six inches or less from the bottom biasing surface 86 of the shaft 16. The handle 24 should move an entire distance of about five inches or less. To enable the scissors action, the tightening throw should proceed through an angle φ of 120° or less. In the tightened position, the grasping surface 84 of the handle 24 is positioned a distance d of from ½ to three inches from the bottom biasing surface 86, which enables a strong single handed grasping force to the fully tightened position. In the preferred embodiment, the tightened squeeze distance d is about 1½ inches. The preferred handle 24 extends for a length of about 3 inches, and pivots through a tightening throw range φ of about 95°.

In the fully tightened position, the grasping portion 57 of the handle 24 extends at a slight angle σ to the shaft axis 50. This grasping angle σ, though not critical, assists in application of a greater squeeze force by the surgeon's normal grip, and also assists in providing clearance for releasing the clamp 22. In the preferred embodiment, the tightened grasping angle σ is about 5°. The bottom side of the grasping portion 57 of the handle 24 provides a spacing 96 of about ¾ of an inch over the top 88 of the shaft 16 for loosening access to the handle 24.

At this size, amount of pivoting and location of the handle pivot axis 43, the grasping surface 57 of the handle 24 is 4 inches away from the bottom biasing surface 86 of the shaft 16 while in the loosened position, and is about ½ inch away from the bottom biasing surface 86 of the shaft 16 when in the tightened position. The handle/shaft combination thus act in conjunction to ergonomically fit the grasp of most surgeons' hands for a single handed, intuitive tightening operation. The surgeon is most commonly standing roughly in line and behind the retractor shaft 16. Just as when cutting a wide cloth a seamstress will lean over a flatly laid cloth and cut away from his or her body, so too the surgeon tightens the clamp 22 with a natural “cutting” orientation, fingers down and thumb up, similar to a handshake position. While the clamp 22 can be readily tightened with a wide variety of single handed orientations, the most common hand orientation naturally coincides with the most common clamp orientation and strongest grip orientation relative to the person tightening the clamp 22.

As best shown in FIGS. 4-6, the arm portion 59 of the handle 24 in the preferred clamp 22 is offset with offset 53 relative to the shaft axis 50, primarily to provide sufficient clearance to maximize the pivot angles θ1 and θ2 and to provide loosened and tightened clearance 55 between the shaft 16 and the handle 24.

As commonly desired, the clamp 22 is capable of being used with a vertical plane containing the shaft axis 50 being oriented perpendicular to the support frame axis 40. For instance, with the shaft/handle of the clamp 22 as shown in the plan view of FIG. 4, the entire clamp 22 is generally balanced and largely symmetrical about a bisecting vertical plane.

When the tightening force is applied in this most common position, the plane defined by the grasping portion 57 tends to orient the tightening force so as to provide the maximum rotational moment on the cam 38 while minimizing any twisting moment of the clamp 22 off the support frame 18. By having a largely balanced, most-common position, the clamp 22 is less likely to twist off the support frame 18 during tightening.

Whether in the tightened position or in the loosened position, the grip portion 57 of the handle 24 always resides vertically above the shaft 16. The shaft 16 is accordingly always in place below the handle 24 for the surgeon's hand to provide equal and opposite squeeze forces on the clamp 22 and hold the clamp 22 from rotating about the support frame 18 during tightening. Orienting the grip portion 57 of the handle 24 and the shaft 16 always in alignment is particularly important when the shaft 16 is at a non-perpendicular angle to the support frame 18, so the tightening forces can be carefully and easily controlled, via single-handed operation, without causing the frame clamp 26 to twist off the support frame 18. To provide this desired orientation of the grip portion 57 of the handle 24, the arm portion 59 of the handle 24 has a vertical S-curve offset 90 (shown in FIG. 3) and a horizontal S-curve to provide the offset 53 (shown in FIG. 6). In the preferred embodiment, the horizontal offset 53 is about 0.8 inches, and the vertical offset 90 from the handle pivot axis 43 to the top of the grip portion 57 in the tightened position is about 2 inches. This preferred handle/cam combination transmits both the tightening force for the clamp 22 and any pivoting moment about axis 58 keeping the handle 24 aligned with the shaft 16.

The “over the top and downward” throw of the handle 24 of the preferred clamp 22 assists in avoiding any interference between the handle 24 and the patient's body or garments. Even if the support frame 18 is positioned very close or in contact with a patient's body or garments, the handle 24 will be accessible from above for its complete throw without interference in any way from the support frame 18 or the patient. In the loosened position (shown in FIG. 3 in dashed lines), the preferred handle 24 projects forward from the frame axis 40 by only a spacing 102 of about 1¼ inches, for a total throw of about 5¼ inches.

In the tightened position of the preferred clamp 22 with the most common orientation of the handle/shaft (in the six o'clock position shown in solid lines in FIG. 4), the grasping portion 57 of the handle is disposed outward from the surgical arena to provide a very low profile clamping arrangement. This is best understood with reference to FIG. 3, wherein:
a) the distal end of the handle 24 has a tightened elevation 92 over the shaft 16 of only about 1 inch, and this highest elevation is spaced distally outward from the frame by a spacing 100 of about 4 inches;
b) the stanchion head 42 projects an elevation 94 of less than ¼ of an inch over the shaft 16, such as a profile elevation 94 of about 0.1 inches; and
c) the bottom surface 86 of the shaft 16 is a distance 98 of only about ¾ of an inch over the support frame 18.
   With this configuration, the tightened clamp 22 virtually never interferes with the surgeon's line of sight or access to the surgical arena.

In the preferred embodiment, the yoke 66 and the C-bearing 68, which bear against the cam 38 during tightening and loosening of the clamp 22, are formed of a strong bearing-grade metal, such as NITRONIC 60 super alloy. The handle 24 is formed by metal injection molding, as further described below. The remaining components may be formed of an appropriately strong sterilizable metal, such as surgical stainless steel.

The double offset handle 24 of the present invention is difficult to cost effectively manufacture out of stainless steel bar stock using bending and machining operations. Rather than using the forming operations for prior art surgical clamp cams and handles, the present invention utilizes a handle 24 and cam 38 which are unitarily formed by metal injection molding.

Powdered metal injection molding is known for other uses. The process begins by combining about 80% metal powder with about 20% binder and additives so as to form a homogeneous granular mixture. The preferred metal powder is 17-4PH stainless steel, but other materials such as 316L, alloy steel and ceramic could alternatively be used. To achieve optimum results, the metal powder has an average particle size of less than or equal to about 15 microns. The binder acts as a viscous carrier, when heated under pressure, to help transport the metal powder through the molding machine's screw feed and into the mold cavity. Binder may include a combination of acetyl, paraffin wax, polypropylene and carnauba wax. Additives may optionally be added to improve the viscosity and moldability of the mixture, as well as to reduce corrosion of the mold tool. Additives may include, for instance, stearic acid and various plasticisers.

The mixture is heated in a conventional metal injection mold press and forced under pressure into a mold cavity. The resultant ejected part is a “green” part, which is considerably larger but of a similar shape to the desired final handle 24 and cam 38. If desired, the green handle/cam may be stored for some period of time after it is removed from mold and before its processing is completed. Care should be taken at this point in handling the green handle/cam due to its extreme brittleness and lack of strength. Rough handling may result in a cracked or broken handle/cam.

The green handle/cam blade is debound by placing parts in a furnace at a temperature of 115° C. with an atmosphere of a catalyst to turn the binder to a vapor. As a result of this debinding step, approximately 85% of the binder and additives are removed from the handle/cam.

The residual binder and additives are removed from the blade during a pre-sintering temperature warmup. A handle/cam part molded from 17-4PH stainless steel is sintered at a temperature of approximately 1360° C. for one hour, in a hydrogen atmosphere. This causes the metal particles to strongly adhere to one another so as to give the molded part its structural integrity. At the end of sintering, the handle/cam is allowed to furnace cool.

Removing the binder and additives from the green handle/cam results in a shrinkage of about 10-30%, and dimensional tolerances may be off on the final part by up to 2%. At this point additional minor forming or machining operations may be performed on the sintered part. However, the design of the preferred clamp 22, and particularly in that the elevation of the adjustment plug 82 and the plunger base 74 are not established until after the handle/cam 24 is manufactured and assembled with the other component parts, allows for a design which is very forgiving in terms of tolerance on the critical cam action. Further, the entire handle 24 is formed with a relatively consistent thickness throughout the part. The opening and consistent near-circular cross-sections in the grip portion 57 in particular enable the handle 24 to provide a wide, flat grip portion 57 while at the same time permitting uniform shrinkage as part of removing the binder and additives from the green part.

The handle/cam is then heat treated for tempering and hardening. For instance, the sintered handle/cam may be held at 480° C. in a hydrogen atmosphere for approximately one hour followed by air cooling, producing a final handle/cam having a hardness of H-900 (equivalent to a hardness of 40-44 on the Rockwell “C” scale).

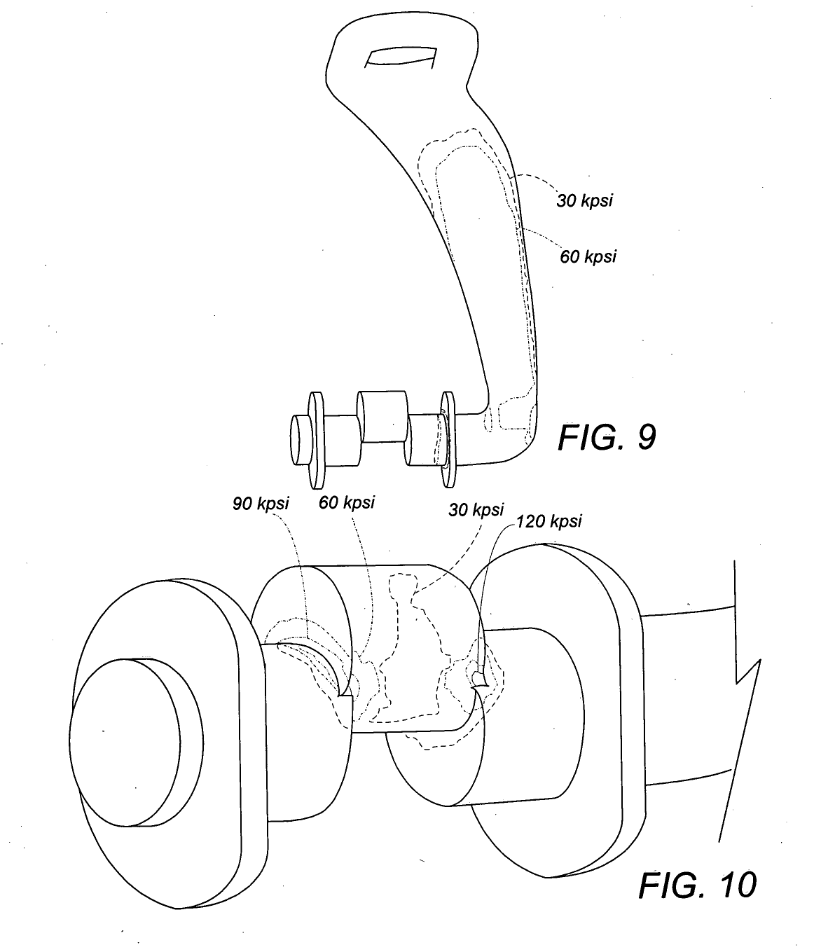

FIGS. 9 and 10 are finite element analysis renderings of the handle/cam of FIGS. 1-8.

In the modeling of FIG. 9, the cam 38 was completely restrained, and a 60 pound force (three to four times the tightening force required after properly elevating the adjustment plug 82) was applied to the grip portion 57 of the handle 24. In the modeling of FIG. 10, the two outside lobes 39 were completely restrained, and a bearing force of 2000 pounds was applied to the middle cam lobe 41. Further, the preferred embodiment has been repetition tested through thousands of tightenings without deformation or damage of the handle 24 or other components of the clamp 22. As shown through this modeling and testing, the preferred design is very robust and strong, without detrimental effects either due to the shrinkage involved with the metal injection molded handle/cam or the dimensional tolerances required of the handle/cam.

Thus it can be seen that the complex shape of the handle 24 provides several distinct advantages during the surgical procedure which are not provided by prior art clamps. Further, the complex shape of the handle 24 is achieved through metal injection molding together with the cam 38 as a single, integrally molded part. The design of the clamp 22 permits the shrinkage and dimensional tolerances of metal injection molding of the handle/cam 24 through both having an adjustable elevation of the base for the cam 38 and by having a handle design of relatively consistent cross-sectional shapes and dimensions, such as including the opening in the grip portion 57 of the handle 24.

Although the present invention has been described with reference to preferred embodiments, workers skilled in the art will recognize that changes may be made in form and detail without departing from the spirit and scope of the invention. For instance, different shapes or offsets in either the handle 24 or cam 38 could be incorporated which utilize the advantages of metal injection molding or other aspects of the present invention once the present invention teaches that metal injection molding is a suitable manufacturing method for handle/cam parts of surgical clamps.

1. A method of making a surgical clamp, comprising:
forming a clamp body, the clamp body having at least a first opening for receiving a first rod, the first opening changing dimensions to receive the first rod in a loosened first clamp position and in a tightened first clamp position;
metal injection molding a handle; and
coupling the handle to the clamp body so the handle is movable to change the dimensions of the first opening.

2. The method of claim 1, wherein the handle is metal injection molded to have a first offset in a first direction and a second offset in a second direction angled with respect to the first direction.

3. The method of claim 1, wherein the handle is metal injection molded to have a grip section and a wedge, with movement of the wedge causing the changing dimensions of first opening.

4. The method of claim 3, wherein the wedge is a cam, wherein rotation of the cam causes the changing dimensions of the first opening.

5. The method of claim 3, wherein the wedge is positioned against a base that, during assembly of the surgical clamp, is adjustable in position relative to the clamp body.

6. The method of claim 5, further comprising: after adjusting the position of the base relative to the clamp body, fixing a bottom position of the base relative to the clamp body.

7. The method of claim 5, further comprising: inserting a spring within the clamp body so the base for the wedge is spring biased against the wedge while the handle is in a loosened position.

8. The method of claim 1,
wherein the clamp body is formed with a second opening for receiving a second rod, the second opening changing dimensions to receive the second rod in a loosened second clamp position and in a tightened second clamp position; and
wherein the handle is coupled to change dimensions of the second opening as well as the first opening.

9. The method of claim 1, wherein the handle pivots through a throw of less than 180° between a loosened stop position and a tightened stop position.

10. The method of claim 1, wherein the handle is metal injection molded to comprise a grip opening therethrough.

11. The method of claim 10,
wherein the handle actuates a wedge to move about a wedge pivot axis, with pivoting movement of the wedge causing the changing dimensions of first opening; and
wherein the grip opening defines a grip plane for receiving an actuating force which, if centered in the grip opening and directed normal to the grip plane, is within a plane normal to the wedge pivot axis and centered relative to the wedge.

12. The method of claim 1, wherein clamp body is formed to comprise:
a bore disposed at an angle to the first opening; and
a tightening pin disposed within the bore, with movement of the tightening pin within the bore causing the tightening pin to change the dimensions of the first opening; and
wherein the handle is coupled to cause movement of the tightening pin within the bore.

13. A method of making a surgical clamp, comprising:
forming a clamp body, the clamp body having at least a first opening for receiving a first rod, the first opening changing dimensions to receive the first rod in a loosened first clamp position and in a tightened first clamp position;
metal injection molding a wedge; and
coupling the wedge to the clamp body so movement of the wedge changes the dimensions of the first opening.

14. The method of claim 13, wherein the wedge is a cam, wherein rotation of the cam causes the changing dimensions of the clamp body.

15. The method of claim 13, wherein the wedge is positioned against a base that, during assembly of the surgical clamp, is adjustable in position relative to the clamp body.

16. The method of claim 15, further comprising: after adjusting the position of the base relative to the clamp body, fixing a bottom position of the base relative to the clamp body.

17. The method of claim 15, further comprising: inserting a spring within the clamp body so the base for the wedge is spring biased against the wedge while the handle is in a loosened position.

8. The method of claim 1, wherein the clamp body is formed with a second opening for receiving a second rod, the second opening changing dimensions to receive the second rod in a loosened second clamp position and in a tightened second clamp position; and wherein the wedge is coupled to change dimensions of the second opening as well as the first opening.

8. the opening being

1. A surgical clamp comprising:
a first clamp member having a frame rod opening for receiving a support frame rod, the frame rod opening being accessible for transverse application onto the first rod, the frame rod opening defining a frame rod opening axis;
a second clamp member having a retractor rod opening for receiving a retractor rod, the retractor rod opening being accessible for transverse application of the retractor rod into the retractor rod opening at an orientation wherein the retractor rod extends in a retractor rod plane above the frame rod, with the retractor rod plane being defined parallel to the frame rod opening axis and the retractor rod opening defining an axis within the retractor rod plane, the second opening changing dimensions to receive the retractor rod in a loosened second clamp position and in a tightened second clamp position, the second clamp member being attached to the first clamp member;
a handle having a handle linkage for moving the second clamp between its loosened second clamp position and its tightened second clamp position, the handle linkage coupled to the second clamp at a position below the retractor rod plane, the handle having a grip portion which is above the retractor rod plane in both the loosened second clamp position and in the tightened second clamp position.

2. The surgical clamp of claim 1, wherein the second clamp member and the handle are both on the same side of the first clamp member.

3. The surgical clamp of claim 1, wherein the grip portion of the handle pivots in a handle plane, wherein the handle plane always substantially contains or is parallel to the retractor rod opening axis.

4. The surgical clamp of claim 1, wherein pivoting of the handle moves the first clamp between its loosened first clamp position and its tightened first clamp position simultaneously with moving the second clamp between its loosened second clamp position and its tightened second clamp position.

5. The surgical clamp of claim 1, wherein when in the loosened second clamp position the second clamp member is pivotable relative to the first clamp member about a pivot axis.

6. The surgical clamp of claim 5, wherein the handle pivots about the pivot axis together with the second clamp member.

7. The surgical clamp of claim 6, wherein the handle moves the first clamp between a loosened first clamp position and a tightened first clamp position and tightens or loosens the pivotable connection between the first clamp member and the second clamp member simultaneously with moving the second clamp between its loosened second clamp position and its tightened second clamp position

8. A surgical clamp comprising:
a first clamp member having a first rod opening for receiving a first rod, the first opening changing dimensions to receive the first rod in a loosened first clamp position and in a tightened first clamp position;
a second clamp member having a second rod opening for receiving a second rod, the second opening changing dimensions to receive the retractor rod in a loosened second clamp position and in a tightened second clamp position, the second clamp member being attached to the first clamp member;
a handle having a handle linkage both for moving the first clamp between its loosened first clamp position and its tightened first clamp position and for moving the second clamp between its loosened second clamp position and its tightened second clamp position, the handle linkage comprising:
a cam member biasing off a bearing surface about a cam center plane, the cam member pivotally rotating about a cam axis;
a shaft portion extending along the cam axis off a single side of the cam member for applying torque to rotate the cam member; and
a handle extension extending to grip portion, the handle extension being bent such that the grip portion extends through or within the cam center plane.

9. The surgical clamp of claim 8, wherein the first rod opening defines a first rod opening axis and the second rod opening defines a second rod opening axis, and, with the surgical clamp oriented such that both the first rod opening axis and the second rod opening axis are horizontal and with the handle in the tightened second clamp position, the handle has a vertical S-curve offset and a horizontal S-curve offset.

10. The surgical clamp of claim 8, wherein the first rod opening defines a first rod axis, wherein the first rod opening is open for receiving the first rod in a direction transverse to the first rod axis, wherein the second opening defines a second rod axis, and wherein the second opening is open for receiving the second rod in a direction transverse to the second rod axis.

11. The surgical clamp of claim 8, wherein when in the loosened position the second clamp member is pivotable relative to the first clamp member about a pivot axis.

12. The surgical clamp of claim 11, wherein the handle pivots about the pivot axis together with the second clamp member.

13. The surgical clamp of claim 12, wherein the handle tightens or loosens the pivotable connection between the first clamp member and the second clamp member simultaneously with moving the first and second clamps between their tightened and loosened positions.

14. A surgical clamp comprising:
a first clamp member having a first rod opening for receiving a first rod, the first rod opening defining a first rod opening axis, the first opening changing dimensions to receive the first rod in a loosened first clamp position and in a tightened first clamp position;
a second clamp member having a second rod opening for receiving a second rod, the second rod opening defining a second rod opening axis, the second opening changing dimensions to receive the second rod in a loosened second clamp position and in a tightened second clamp position, the second clamp member being attached to the first clamp member;
a handle both for moving the first clamp between its loosened first clamp position and its tightened first clamp position and for moving the second clamp between its loosened second clamp position and its tightened second clamp position, the handle having a grip portion defining a plane of tightening force application, wherein a tightening force applied to the grip portion normal to the plane of tightening force application intersects the second clamp opening axis.

15. The surgical clamp of claim 14, wherein, with the handle in the tightened position, the plane of tightening force application is parallel to the second rod opening axis.

16. The surgical clamp of claim 14, wherein the grip portion contains a central hole therethrough, normal to the plane of tightening force application.

17. The surgical clamp of claim 14, wherein when in the loosened position the second clamp member is pivotable relative to the first clamp member about a pivot axis.

18. The surgical clamp of claim 17, wherein the handle pivots about the pivot axis together with the second clamp member.

19. The surgical clamp of claim 18, wherein the handle tightens or loosens the pivotable connection between the first clamp member and the second clamp member simultaneously with moving the first and second clamps between their tightened and loosened positions.
